(19) United States
(12) Patent Application Publication
(10) Pub. No.: US 2008/0221520 A1
(43) Pub. Date: Sep. 11, 2008

(54) Positioning System for Percutaneous Interventions

(75) Inventors: Markus Nagel, Nurnberg (DE); Gero Schnutgen, Erlangen (DE); Ralf Petzold, Erlangen (DE)

Correspondence Address: LERNER GREENBERG STEMER LLP, P O BOX 2480, HOLLYWOOD, FL, 33022-2480, US

(73) Assignee: CAS Innovations AG, Erlangen (DE)

(21) Appl. No.: 12/048,564

(22) Filed: Mar. 14, 2008

Related U.S. Application Data
(63) Continuation of application No. PCT/EP2006/008960, filed on Sep. 14, 2006.

(30) Foreign Application Priority Data
Sep. 14, 2005 (DE) 10 2005 044 033.9

(51) Int. Cl. A61M 5/00

(52) U.S. Cl. 604/116

(57) ABSTRACT

A positioning system for percutaneous interventions, which is useful with clinical operating procedures and permits rapid and precise insertion of a needle or other medical instrument into a patient's body. Positioning is accomplished by using a combination of navigation software and a reference frame and needle or instrument holder.

The invention relates to a positioning system for percutaneous interventions. Furthermore, the invention relates to a reference frame and an instrument mount for use in such a positioning system. Finally, the invention relates to a computer program for such a positioning system.

Image-assisted interventions, in particular CT-assisted interventions, are nowadays part of clinical routine. In contrast to an invasive surgical treatment, minimally invasive image-assisted interventions enable the user to work with minimal injury to the patient. This does not just reduce the clinical costs, it also reduces the risk of complications and has a positive cosmetic effect.

However, the accuracy and speed with which a puncture needle is positioned in the patient's body depends to a great extent on the radiologist's ability. In particular, such a procedure requires a high degree of experience. Even finding a suitable entry point is often arbitrary. Therefore, a large number of control scans are normally necessary in order to determine the needle position and to correct it, if appropriate, until the needle tip is situated at the desired target point. This is necessary particularly in those applications in which an incorrect position of the needle can lead to life-threatening states for the patient. The frequent control scans not only prolong the duration of the intervention, but also increase the radiation dose for the patient.

One object of the present invention is to improve the positioning of medical instruments in percutaneous interventions.

This object is achieved by means of a positioning system according to Claim 1. Accordingly, a positioning system for percutaneous interventions is provided, comprising a reference frame for arrangement in a defined position relative to a patient, wherein the reference frame is formed in such a way that it is possible to determine the position thereof in a first reference system and in a second reference system, comprising an instrument mount for receiving and/or holding a medical instrument, in particular a needle, and/or comprising a medical instrument, wherein the instrument mount and/or the medical instrument is formed in such a way that it is possible to determine the position thereof in the second reference system, and comprising a data processing unit having a) an input module, designed for receiving a patient data record provided by an imaging system and for receiving an apparatus data record provided by a tracking system, wherein the patient data record contains patient data, in particular image data, in the first reference system and data regarding the position of the reference frame in the first reference system, and wherein the apparatus data record contains data regarding the position of the reference frame in the second reference system and data regarding the position of the instrument mount and/or of the medical instrument in the second reference system, b) a registration module, designed for carrying out an automatic image-to-patient registration with the aid of the data contained in the patient data record and in the apparatus data record, and c) a planning module designed for planning a trajectory from an entry point on the patient to a target point in the patient.

Furthermore, this object is achieved by means of a reference frame according to Claim 12. The reference frame for use in a positioning system is designed for arrangement in a defined position relative to a patient, comprising a number of first marking elements for determining its position in a first reference system and comprising a number of second marking elements for determining its position in a second reference system, wherein the first marking elements are designed for determining a position with the aid of an imaging method, in particular computer tomography, and wherein the second marking elements are designed for determining a position with the aid of an optical or electromagnetic tracking method. It is particularly advantageous if the reference frame can be fitted to or on the patient. In this case, an additional fixing of the patient can be achieved by pressing the reference frame onto the patient's body. As an alternative to this, the reference frame is arranged relative to the patient without there being any mechanical contact with the patient. The risk of transmission of germs or the like is very low in this case.

Furthermore, this object is achieved by means of an instrument mount, in particular needle mount, according to Claim 15. The instrument mount for use in a positioning system comprises a receiving or holding device for fixing a medical instrument, in particular a needle, and comprises a number of second marking elements for determining its position in a second reference system, wherein the second marking elements are designed for determining a position with the aid of an optical or electromagnetic tracking method. The instrument mount preferably has two rotary joints for simple and precise orientation.

Furthermore, this object is achieved by means of a medical instrument according to Claim 17. The medical instrument, in particular a needle, comprises a number of second marking elements for determining its position in a second reference system, wherein the second marking elements are designed for determining a position with the aid of a preferably optical or electromagnetic tracking method.

Furthermore, this object is achieved by means of a computer program for a positioning system for percutaneous interventions according to Claim 18. Accordingly, it is provided that the computer program has: computer program instructions for receiving a patient data record provided by an imaging system and for receiving an apparatus data record provided by a tracking system, wherein the patient data record contains patient data, in particular image data, in a first reference system and data regarding the position of the reference frame in the first reference system, and wherein the apparatus data record contains data regarding the position of the reference frame in the second reference system and data regarding the position of the instrument mount and/or a medical instrument in the second reference system, computer program instructions for carrying out an automatic image-to-patient registration with the aid of the data contained in the patient data record and in the apparatus data record, and computer program instructions for planning a trajectory from an entry point on the patient to a target point in the patient, if the computer program is executed on a computer.

One fundamental concept of the invention consists in planning the access path before the actual intervention and in ensuring a precise and fast orientation of the medical instrument and a defined advance with the aid of suitable devices. This enables a computer-aided navigation for percutaneous interventions, and this navigation can be applied in particular in the area of interventional radiology. In this case, the term navigation is understood to mean determining the position by tracking. Furthermore, the term navigation is understood to mean planning the access path to the target point. Finally, the term navigation is also understood to mean guiding a medical instrument to said target on the planned access path.

A further fundamental concept of the invention consists in the combination of a navigation software with a specific instrument mount and/or a specific medical instrument. With this combination, an accurate orientation of the instrument, preferably a needle or the like, in accordance with the planned trajectory is possible within a few seconds. In accordance with a further fundamental concept of the invention, moreover, a fully automatic image-to-patient registration is made possible by the use of a specific reference frame. This likewise contributes to very fast provision of the information required for the intervention, and therefore overall to a shortening of the treatment duration.

The novel image-assisted navigation method enables minimally invasive interventions in which even extremely small target regions within the body can be reliably reached with pinpoint accuracy. In this case, the positioning system according to the invention can be completely integrated into the clinical procedural environment.

The invention can be used in all image-assisted interventions and therapies requiring a percutaneous advance of a needle to a specific anatomical position in the patient. Fields of application are, inter alia, biopsy or puncture for removing tissue in need of clarification (for example thorax, abdominal, spine, hip, knee, etc.), vertebroplasty, periradicular therapy and radio-frequency ablation.

The positioning system according to the invention makes it possible to reduce the number of control scans and thus the duration of the intervention and the radiation burden for the patient. The reduction of the duration of the intervention, in particular, is of great importance when a rapidly dissolving contrast medium is used.

Preferably, not a single marking element is situated directly on the patient's body. Nevertheless, in most cases a precise intervention is possible without general anaesthetic. The burden for the patient can therefore be significantly reduced in comparison with solutions known from the prior art. This can be achieved, inter alia, by the use of a highly effective patient fixing system which minimizes unpredictable movements by the patient relative to the reference frame, which might influence the image-to-patient registration. Risks associated with movements by the patient can thereby be significantly reduced in comparison with known methods.

In contrast to conventional positioning systems or positioning aids, such as lasers or skin markers, the present positioning system is adapted to a radiologist's needs and can therefore be used as an ideal tool for image-assisted percutaneous interventional methods.

Advantageous embodiments of the invention are specified in the subclaims.

Thus, in accordance with one particularly preferred embodiment of the invention it is provided that the data processing unit has a navigation module designed for visualizing the instrument mount and/or the medical instrument in the patient data record before and/or during the intervention. In other words, therefore, according to the invention it is not just possible to check the position of the medical instrument outside the patient's body. With the invention there is additionally the possibility of visualizing the needle advance in the patient data record in real time and of thus controlling the introduction of the needle into the patient's body and the movement of the needle. As a result, the number of control scans and therefore the radiation burden for the patient can be significantly reduced in comparison with conventional techniques.

In accordance with a further particularly preferred embodiment of the invention, the use of an optical and/or electromagnetic tracking system is provided. With the use of an optical tracking system, it is possible to track the medical instruments used outside the patient. If, in addition or as an alternative to this, an electromagnetic tracking system is used, it is possible to track the medical instruments used in the patient's body as well.

In accordance with a further particularly preferred embodiment of the invention use is made of a patient fixing system for fixing the patient, which system is preferably based on a vacuum principle. The extensive immobilization of the patient that can be achieved as a result of this enables the advantages of the invention to be utilized particularly well.

Further advantages and embodiments of the invention are explained in more detail below with the aid of exemplary embodiments. In this case:

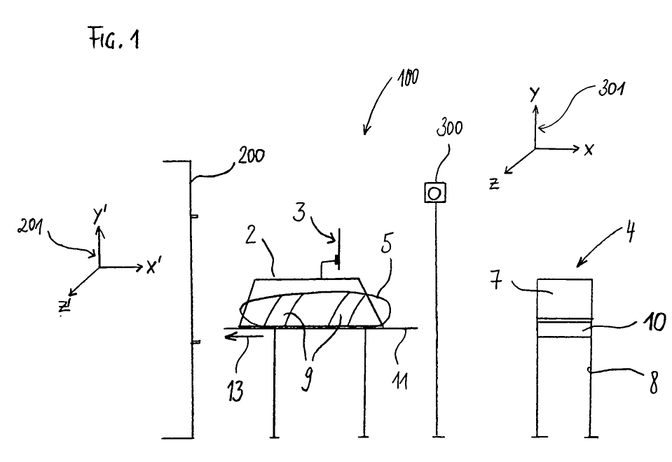

FIG. 1 shows an overview representation of a positioning system,

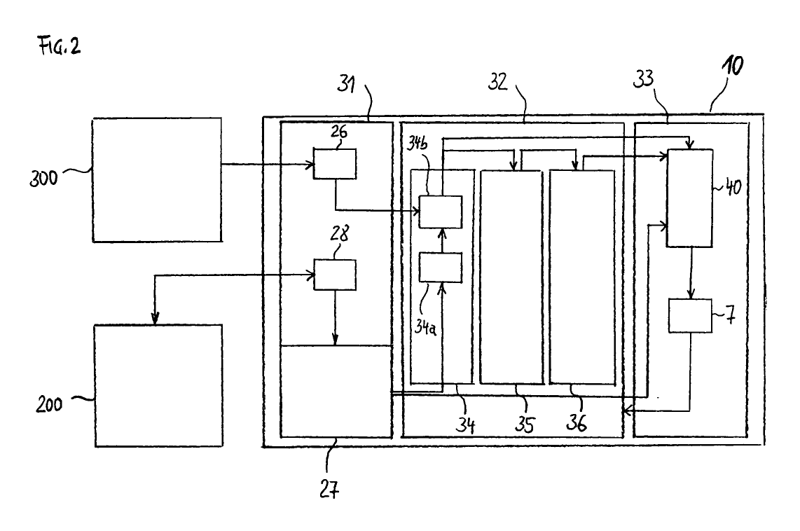

FIG. 2 shows a block representation of a positioning system,

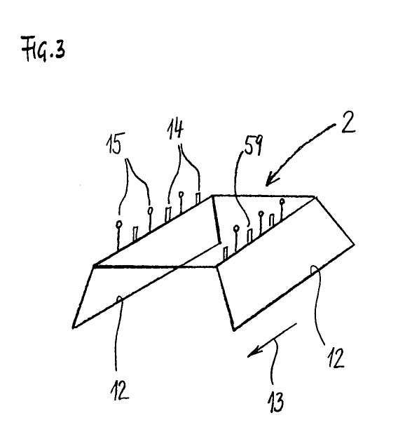

FIG. 3 shows a reference frame,

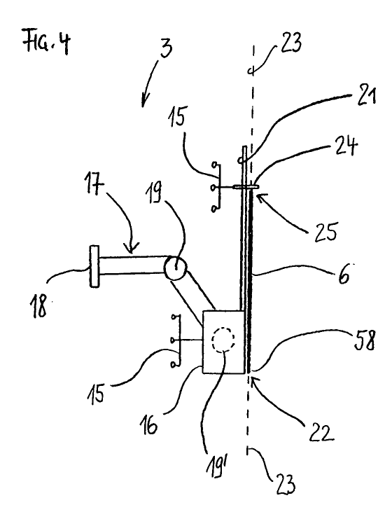

FIG. 4 shows a needle mount,

FIG. 5 shows a screen representation of the trajectory planning,

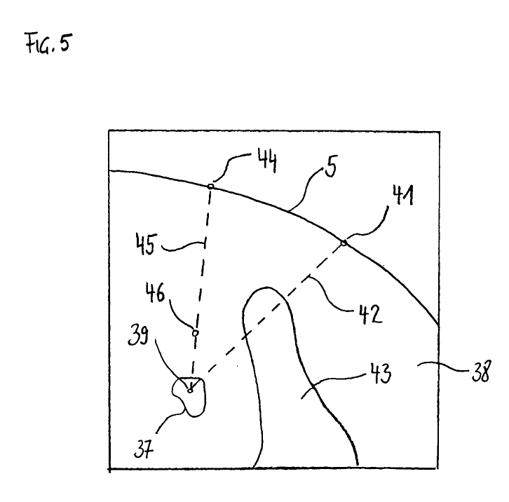

FIG. 6 shows a first screen representation during the orientation of the needle mount,

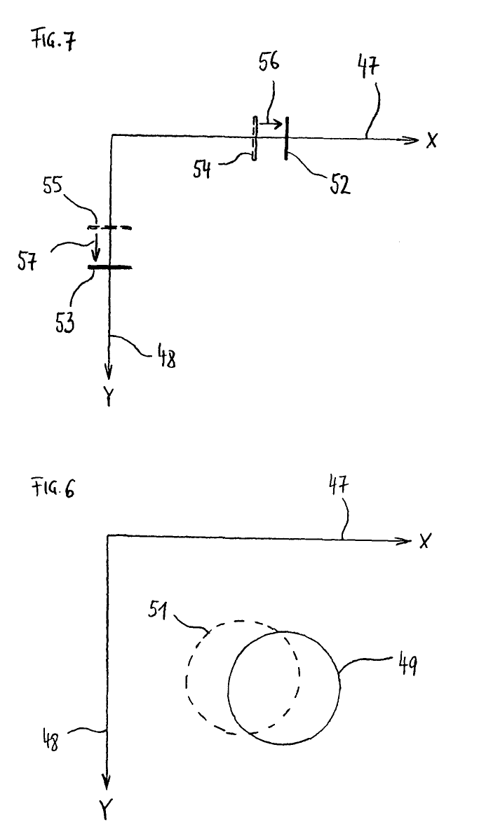

FIG. 7 shows a second screen representation during the orientation of the needle mount,

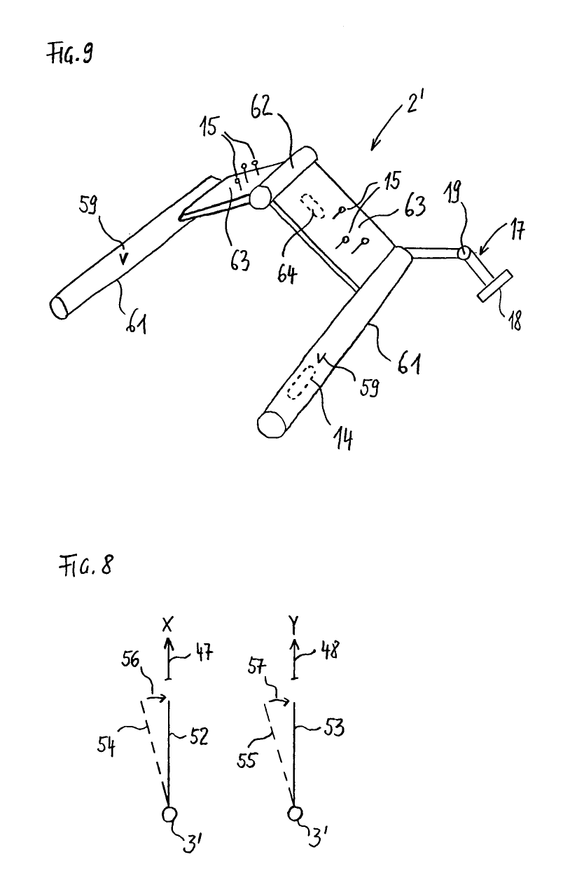

FIG. 8 shows an alternative second screen representation during the orientation of the needle mount, and

FIG. 9 shows a further reference frame.

All of the figures show the invention merely schematically and with its essential constituents and also in part in greatly simplified fashion.

With the aid of the technique described below, the clinical objective will be achieved of sticking a needle percutaneously into a patient and advancing it along a previously planned access path to a defined target point in order to begin a corresponding therapy. The intervention takes place extremely precisely and rapidly in this case.

As illustrated in FIGS. 1 and 2, the positioning system 100 according to the invention comprises a reference frame 2 and a needle mount 3. Furthermore, the positioning system 100 comprises a data processing unit 4. The data processing unit 4 is designed for carrying out all the steps in accordance with the method described here which are associated with the processing of data. The data are, in particular, data concerning the patient 5 to be treated, such as image data, for example, and data concerning various components of the positioning system 100, in particular position data of the needle mount 3, of the reference frame 2 and, if appropriate, of the needle 6. The data processing unit 4 preferably has a number of functional modules that are explained in more detail further below, wherein each functional module is designed for carrying out a specific function or a number of specific functions in accordance with the method described. The functional modules can be hardware modules or software modules. In other words, the invention, in so far as the data processing unit 4 is concerned, can be realized either in the form of computer hardware or in the form of computer software or in a combination of hardware and software. In so far as the invention is realized in the form of software, the functions described below are realized by computer program instructions, if the computer program is executed on a computer. In this case, the computer program instructions are realized in any desired programming language in a manner known per se and can be provided to the data processing unit 4 in any desired form, for example in the form of data packets that are transmitted via a computer network, or in the form of a computer program product stored on a floppy disk, a CD-ROM or some other data carrier.

In the exemplary embodiment discussed here, the data processing unit 4 is a standard personal computer (PC) 10 with a touch-sensitive screen (touch screen) 7 serving as user interface. A navigation software is executed on the PC 10. The PC 10 is preferably accommodated in a small movable carrier rack 8 that can be moved as required in a simple manner within the operating theatre. Furthermore, a patient fixing system 9 is employed, which is indicated merely schematically in FIG. 1 and which ensures that, in particular, external movements of the patient 5 are suppressed as well as possible.

For image acquisition, the positioning system 100 is connected to a computer tomograph (CT scanner) 200. The CT scanner 200 is for example a scanner of the type Sensation 64 from Siemens Medical Solutions (Germany). As an alternative, instead of the CT scanner 200, it is also possible to use a 3D C-arc system or a magnetic resonance system for image acquisition. Furthermore, other imaging methods can also be used, in principle. All that is important here is that a volume representation, that is to say a three-dimensional image data record of the patient 5 can be created thereby. The selection of the suitable imaging method is dependent in particular on the clinical issue. The use of a CT scanner 200 is particularly advantageous, however, since this can cover a large proportion of the possible applications.

The positioning system 100 is furthermore connected to a tracking system 300. In this case, the optical tracking system 300 can be regarded as part of the positioning system 100. However, it is likewise possible to regard the reference frame 2, the needle mount 3 and the data processing unit 4 as the actual “core” positioning system 100 which interacts with a tracking system 300.

In the exemplary embodiment illustrated, the tracking system 300 is a passive optical tracking system, which detects the position of passive marking elements in the space. By way of example, a system of the type POLARIS from NDI, Canada can be used. In this case, a special camera is used for recording three-dimensional digital photographs of the patient and the apparatus (reference frame and needle mount/needle). Instead of a passive optical tracking system, however, it is also possible to use an active optical tracking system, in which active infrared markers or light-emitting diodes are used as marking elements. All that is important here is that the position of marking elements in a three-dimensional space can be detected with the aid of the tracking system. Therefore, instead of the optical tracking system it is also possible to use an electromagnetic tracking system or the like.

Minimally invasive interventions could be carried out under local anaesthetic in many cases. On account of the movements of the patient 5, however, the intervention is often performed under general anaesthetic in the case of the techniques known from the prior art. With the aid of a suitable patient fixing system 9, the positioning system according to the invention enables interventions under local anaesthetic.

In this case, inter alia the fixing system “Fixierungssystem” from Medical Intelligence (Germany) has proved to be particularly suitable as a patient fixing system. However, it is also possible to use other patient fixing systems. All that is important here is that a highly accurate fixing of the patient 5 relative to the reference frame 2 is ensured.

In order to prepare for the intervention with the aid of the fixing system, the body of the patient 5 lying on a CT table 11 in a vacuum mat is covered with air-permeable cushions. A plastic film is then placed over the patient 5 and the cushions and a pump is used to suck the air from the cushions and the vacuum mat. As a result, the cushions and the vacuum mat become hard and match the body contours of the patient 5. Since involuntary movements of the patient are thereby completely prevented, the system can be operated using local anaesthetic. Operation under general anaesthetic, as is required in known systems, with the attendant risks for the patient, can be obviated. In addition to reducing the movements of the patient, the use of the patient fixing system 9 also enables the patient to be returned to the initial position of the patient in a reproducible manner after an unpredictable movement of the patient.

The reference frame 2 is subsequently positioned. The reference frame 2 is composed of a rack which is preferably produced from a carbon or plastic material and is therefore free of artefacts with respect to the imaging and also extremely robust and easy to maintain, cf. FIG. 3. In this case, the reference frame 2 is preferably formed in such a way that it can be arranged relative to the patient 5 at any desired location of the patient 5 without there being any mechanical contact with the patient 5. In other words, reference frame 2 and patient 5 are completely decoupled from one another. This makes it possible to ensure a high degree of freedom from germs. By way of example, it is possible to provide the reference frame 2 with a sterile covering. Even work on open wounds or the like is therefore possible without any problems.

In the exemplary embodiment shown in FIG. 3, the reference frame 2 is embodied in skeletal fashion in the form of a slide. In this case, the reference frame 2 bears with its lower longitudinal struts 12, which serve as bearing surfaces, on the CT table 11 and completely straddles the body of the patient 5 without touching the latter. During a CT scan, the CT table 11 is moved in the longitudinal direction 13 towards the CT scanner 200 and travels together with the body of the patient 5 and the reference frame 2 into the CT scanner 200. The reference frame 2 is therefore made flat enough that travel into the CT scanner 200 is possible without any problems.

In a further embodiment of the invention (not illustrated), the reference frame is configured in such a way that it can be fixed directly to or on the patient. The reference frame then no longer has any mechanical connection to the CT table 11. Moreover, by fitting a number of markings (not illustrated) on the patient 5, for example on the skin of the patient 5, it is possible to check whether the position of the reference frame has changed during the measurement.

As illustrated in FIG. 3, first marking elements (CT markers) 14 are fixed to the reference frame 2. Said CT markers 14 are configured in such a way that they can be automatically recognized in the CT images produced by the CT scanner 200. In particular the CT markers 14 for this purpose have a particularly high HU (Hounsfield Unit) value. In this case, the CT markers 14 can be mounted—as illustrated—on the surface of the reference frame 2 or else be arranged in the interior of the reference frame (cf. FIG. 8).

Furthermore, second marking elements (optical markers) 15 are fixed to the surface of the reference frame 2. The optical markers 15 are made reflective in such a way that they can be recognized by a passive optical tracking system 300. The use of reflective balls as optical markers 15 is particularly advantageous.

Both CT markers 14 and optical markers 15 are in each case fitted in a defined geometrical arrangement on the reference frame. They therefore form in each case a DRF (Dynamic Reference Frame) system for determining the coordinate system 201 of the CT scanner 200 and respectively the coordinate system 301 of the optical tracking system 300 and thus the basis of an image-to-patient registration. Since the CT markers 14 and optical markers 15 are fixedly fitted to the reference frame 2, their position with respect to one another is defined. This position information is known to the positioning system 100, such that it is possible to adjust the two coordinate systems, namely the patient coordinate system 201 on the basis of the CT data of the CT scanner 200 on the one hand, and the coordinate system 301 on the basis of the data of the optical tracking system 300, on the other hand.

The needle mount 3 is embodied in such a way that any desired medical instrument, such as, for example, a biopsy needle or a cannula, can be fixed, and demounted again, rapidly and in a simple manner. As a result, biopsy needles or cannulae can be integrated into the system in a simple manner even during the intervention. In the exemplary embodiment, the instrument is a puncture needle 6. The needle 6 is held in the needle mount 3 in the region of its proximal end by means of a receiving or holding device 16. The needle mount 3 furthermore has a mounting arm 17, to one end of which the receiving or holding device 16 is fixed. Said mounting arm 17 can be fixed by its other end to the reference frame 2 by means of a fixing flange 18. However, the mounting arm 17 can for example also be mounted on the CT table 11 of the CT scanner 200.

The needle mount 3 has two rotary joints 19, 19′ that can be actuated independently of one another, whereby the needle mount 3 can be oriented to the planned trajectory rapidly and accurately. In this case, one of the rotary joints 19 is formed as part of the mounting arm 17, while the other rotary joint 19′ is provided in or on the receiving or holding device 16. Both rotary joints 19, 19′ can be fixed in any desired positions. The needle mount 3 therefore preferably has six degrees of freedom, such that it can be positioned in a simple manner in the vicinity of the patient 5 and in particular in the vicinity of the entry site. A bearing rail 21 extends from the receiving or holding device 16, in which a puncture needle 6 is always held in the exemplary embodiments illustrated, the needle 6 being guided on said bearing rail.

In an alternative embodiment, the needle mount 3 and/or the reference frame 2 are fixed to a hydraulic mounting arm (not illustrated). It is then particularly simple to arrange both needle mount 3 and reference frame 2 at any desired position on the patient 5.

Optical markers 15 are also fitted to the needle mount 3. In the exemplary embodiment shown, said optical markers correspond to those optical markers 15 as already used in the case of the reference frame 2. In this case, the optical markers 15 are fitted to the needle mount 3 in such a way that both the position of a rotation point 22 of the needle mount 3 and the position of the needle axis 23 running through the rotation point 22 are known to the positioning system 100 through transmission of the corresponding position information of the markers 15. In this case, the rotation point 22 is the point around which the needle mount 3 is rotated later during the orienting process. In addition, the needle 6 itself is assigned a number of further optical markers 15 in order to be able to determine the later penetration depth of the needle 6. In the present exemplary embodiment, the further optical markers 15 are situated on a bearing element 24, which bears on the distal end 25 of the needle 6 and is arranged in displaceable fashion on the bearing rail 21 in such a way that when the needle 6 penetrates into the body of the patient 5, at the same time the bearing element 24 and thus the optical markers 15 can also be displaced or become displaced themselves.

Both the CT markers 14 and the optical markers 15 are in each case fixed in a defined geometrical arrangement on the reference frame 2 and on the needle mount 3, such that position determination in the three-dimensional space or in the patient data record is unambiguously possible on the basis of the markers 14, 15. Preferably, at least three markers 14, 15 of one type are in each case provided for this purpose on each device. The number of CT markers 14 provided on the reference frame 2 is preferably higher, however, in order that an unambiguous assignment is possible even when, rather than the entire reference frame 2, only a portion of the reference frame 2 and therefore also only a portion of the CT markers 14 are detected by the CT scan.

The optical tracking system 300 serves to ascertain the position of the reference frame 2 and of the needle mount 3 in the operating theatre with the aid of the optical markers 15. For this purpose, the position of the needle mount 3 relative to the reference frame 2 is determined. All 3D coordinates required are communicated from the optical tracking system 300 to the PC 10 using the serial PC interface 26.

In order to visualize a medical instrument, for example the needle 6, in the CT images of the patient 5, an image-to-patient registration is necessary. If the patient 5 is fixed, the reference frame 2 is therefore positioned in direct proximity to the planned entry point before the first CT scan. In this case, the reference frame 2 is positioned in particular in such a way that as many CT markers 14 as possible are situated in the vicinity of the entry point.

Before the first CT scan, the positions of the individual devices with respect to one another and with respect to the patient 5 are controlled in order to ensure that a correct evaluation is possible later. This control primarily serves to avoid unnecessary repetitions of the CT scan and thus unnecessary radiation burdens for the patient 5.

During the CT overview scan that then follows, a field of view is determined in such a way that preferably all of the CT markers 14 are situated within the field of view during the CT scan. At least three CT markers 14 must be situated in the field of view, however, in order that an unambiguous position determination is possible.

The CT scanner 200 reconstructs a 3D representation of the patient 5 from the scan data. After the CT scan has been carried out and the 3D representation has been created, the CT images are transmitted in the form of slice images to the positioning system 100. The transmission and the loading of the CT images from the CT scanner 200 are preferably effected fully automatically. However, it is likewise possible for a preselection to be made by the user, for example a radiologist, before the transmission of the CT images.

For the data exchange between the positioning system 100 and the CT scanner 200 via the hospital's internal network, a communication software is provided, which enables image transmission, including the associated verification, storage, enquiry and retrieval services, using a DICOM (Digital Imaging and Communications in Medicine) network 27 with the aid of a TCP/IP connection 28. Said communication software is implemented as a background process and set up in such a way that CT images are received as soon as the navigation software is executed.

The use of standard communication connections such as the TCP/IP network connections of the positioning system 100 with the CT scanner 200 and the DICOM protocol enable a manufacturer-independent and convenient image transfer in both directions.

The software CAPPA IRAD, developed by the patent applicant, is preferably employed as navigation software. The navigation software is structured in modular fashion and has, inter alia, an input and output module 31, a computation module 32 and a display module 33. In this case, the input and output module 31 is designed for receiving and transmitting data to connected devices or systems and the display module 33 serves for communicating information to the user. For this purpose, the display module 33 comprises a control unit 40 designed for driving the touch-sensitive screen 7, wherein a graphical user interface (GUI) is used for user guidance and interaction with the user. The computation module 32 has a number of submodules, inter alia a registration module 34, a planning module 35 and a navigation module 36. These modules are designed in the broadest sense for processing data, wherein the registration module 34 is designed, inter alia, for carrying out the image-to-patient registration, the planning module 35 is designed, inter alia, for planning a trajectory describing the access path, and the navigation module 36 is designed, inter alia, for navigating the needle 6 in the body of the patient 5. Furthermore, the navigation software comprises a number of further functional modules (not illustrated) which are designed for data processing in the sense of the invention.

The screen 7 driven by the display module 33, in the same way as optional connected further input devices, such as a computer mouse, an external keyboard or the like, is connected to the PC 10 and designed in such a way that data inputting and/or control of the navigation software or of the positioning system 100 and preferably also of the systems (in particular CT scanner 200 and tracking system 300) connected to the positioning system 100, and thus of the entire navigation method is possible with the aid of said input devices.

After the CT images have been received, they are visualized in sectional image views (coronal, sagittal, transverse) by means of the control unit 40 in the display module 33 of the navigation software. Prior to visualization, all of the CT images are checked by the positioning system 100 by means of a further functional module with regard to the existence of correspondence in respect of the patient data. This prevents image data of another patient from being incorrectly displayed. Preferably, a further functional module also provides for checking the transmitted number of images in order to check a complete data transmission from the CT scanner 200 to the positioning system 100 and to ascertain a possible failure of the hospital's communication network in good time. Furthermore, the CT images are checked by the user and stored by means of a further functional module in the positioning system 100. With the aid of the stored CT images, a rapid overview of the CT data is possible later during the intervention.

Following the transmission of the CT images to the positioning system 100 via the TCP/IP interface 28, a marker recognition algorithm integrated in the registration module 34 is preferably executed automatically, said algorithm recognizing the CT markers 14 in the patient data record and determining the marker mid points with an accuracy in the sub-voxel range. For this marker recognition, a specific marker recognition module 34a is provided within the registration module 34. For the image-to-patient registration, the coordinates of the CT markers 14 in the patient coordinate system 201 and the coordinates of the CT markers 14 in the coordinate system 301 of the optical tracking system 300 are subsequently adjusted with one another by means of the registration module 34. A registration matrix is generated in this case. For this purpose, the registration module has a specific adjustment module 34b. After this adjustment of the two marker groups there is a fixed relationship between the CT images and the patient 5. The entire registration process preferably takes place fully automatically in this case. If the automatic adjustment is not successful, an error message ensues via the display module 33, which is driven by the registration module 34 for this purpose. An adjustment of the individual marker positions with respect to one another can then also be effected manually by the user.

The access path is then planned with the aid of the planning module 32. The user defines a trajectory for this purpose. This is done in the case of a rectilinear trajectory, in the simplest case, by defining a target point and an entry point in the 3D representation of the patient 5.

FIG. 5 shows an example of such planning on the basis of a screen representation. The representation shows part of the body of the patient 5 with a target region 37, from which a tissue sample, for example, is to be taken. Said target region 37 lies within a first tissue type 38. The user firstly determines the target point 39 and a first entry point 41, whereby a trajectory 42 is defined. In this case, however, this first trajectory 42 runs through a second tissue type 43 of the patient 5, which is not intended to be damaged. Therefore, the user selects a second entry point 44 with respect to the same target point 39, said second entry point being spaced apart sufficiently from the first entry point 41. The resultant second trajectory 45 runs from the entry point 44 to the desired target point 39 entirely through the first tissue type 38 and can therefore be used for the actual intervention.

In other words, the trajectory 45 is planned on the basis of the representation of the patient data, or to put it another way in the patient data record. In this case, entry and target points 44, 39 are defined either by means of a computer mouse or with the aid of the touch-sensitive screen 7. In this case, the target point can be situated in soft tissue or else on or in a bone. Trajectories that do not run in rectilinear fashion can also be planned.

Furthermore, a number of checkpoints 46 can be defined by the user. If the needle 6 reaches one of the checkpoints 46 during the intervention, the CT scanner 200 can be used to perform a control recording in order to check the needle position. It goes without saying that carrying out the control scans is not bound to the reaching of the checkpoints 46. Rather, CT scans can be carried out at any desired points in time.

In this case, the GUI or the control unit 40 of the navigation software is programmed in such a way that the navigation software can be used intuitively by the user. In the planning module 35, besides the standard slice image views, a multiplicity of further planning functions are realized, for example oblique sectional images and the precise planning of trajectories with sub-voxel accuracy. Oblique sectional images, that is to say sectional images that run obliquely through standard slice image views, are in this case calculated by the planning module 35 of the positioning system 100 in the patient data record.

It is furthermore possible to plan any desired oblique trajectories. In other words, the planning module 35 permits not only the planning of trajectories 45 that lie in one or two transverse CT slices, but also the planning of those trajectories that run obliquely through the entire scanned 3D volume of the patient data record. Obliquely running trajectories in the patient data record can be represented with the aid of the obliquely running sectional images. Consequently, surrounding structures on the trajectory can be assessed at a glance.

In other words, the access path is calculated by the planning module 35 and represented three-dimensionally on the screen 7 by the control unit 40 of the display module 33. It can therefore be checked in a very simple manner by the user. In this case, it is possible, for example, to ascertain whether the needle that is subsequently to be introduced will have undesirable contact with tissue parts, for example internal organs, or bone on its way to the target point. A multiplicity of different control views can be carried out for this purpose. Inter alia, a view is possible in which the access path is traveled from the point of view of the needle 6. Other views are standard slice image views, freely definable slice image views, and fixed needle slice images. As necessary, by means of a virtual change of the entry site it is possible to modify the course of the trajectory in the planning module 35 and to recheck the access path.

What is advantageous about this type of trajectory planning is that any desired number of trajectories can be planned virtually, without patient's tissue actually being damaged. Thus, the user can find an access path which is optimal for the respective intervention or the respective therapy, on the one hand, and to the patient 5, on the other hand.

In order to prevent the patient 5 from gaining knowledge during the planning of the access path from a possible conversation among the persons involved, an exemplary embodiment of the invention that is not illustrated advantageously provides for carrying out the trajectory planning at a spatially separate planning station, which can preferably be installed in a separate room. For this case, part of the navigation software, in particular the planning module 35, is embodied in such a way that it can also be executed separately from the rest of the modules. In this case, the data transmission between the modules within the navigation software remains possible in an unchanged manner, for example via a direct data connection between the computers executing the respective modules.

The subsequent orientation of the needle 6 in accordance with the planned trajectory and the navigation of the needle 6 in the patient 5 can be effected in two different ways.

In a first exemplary embodiment, the needle mount 3 is only oriented to the previously planned trajectory 45, without the needle length being displayed by the display module 33 in the patient data record. Instead, the needle 6 is represented outside the patient 5 and the navigation module 36 only guides the user during the orientation of the needle mount 3. During the needle advance, it is possible to use optical markings, for example colour codes, on the needle 6 in order to obtain information about the penetration depth.

The orientation itself is effected in two partial steps here. Firstly, the user moves the needle mount 3 into the vicinity of the entry site provided. In this case, said user is guided by the navigation module 36 by virtue of the representation of the position of the needle mount 3 in the patient data record on the screen 7. The process of leading the needle mount 3 to the entry point 44 is effected using the mounting arm 17 and the rotary joints 19, 19′ and usually takes less than 10 seconds. The first step is concluded by the user putting the rotation point 22 of the needle mount 3 onto any desired location of the trajectory 45 represented on the screen 7.

FIG. 6 illustrates a screen representation such as is presented to the user by the navigation module 36 with the aid of the control unit 40 at this place in the method. In a two-dimensional coordinate system spanned by an X-axis 47 and a Y-axis 48, the position of the trajectory 45 is represented as a desired position of the needle mount 3 in the form of a first circle 49. Furthermore, the actual position of the rotation point 22 of the needle mount 3 is represented, likewise in the form of a circle 51. In this case, the desired position is represented by a solid line and the actual position is represented by a broken line. Different-coloured representations are preferably used on the screen 7 in order to identify the desired and actual positions. The first step is concluded when the second circle 51 lies on the first circle 49.

Afterwards, using the two remaining spatial axes, the needle mount 3 is oriented in such a way that the needle axis 23 lies on the planned trajectory 45. In this case, the navigation module 36 provides the user with important information as to how the needle mount 3 has to be moved by means of the two rotary joints 19, 19′. In particular, information about the current distance to the entry point 44 and information regarding the correct entry angle are output via the screen 7. With some practice, the orientation of the needle mount 3 is effected in less than 10 seconds.

FIG. 7 illustrates a further screen representation such as is presented to the user in this situation. The desired position 52 of the needle axis 23 on the X-axis 47 and the desired position 53 of the needle axis 23 on the Y-axis 48 are respectively represented in the coordinate system already described. Furthermore, the actual position of the needle axis 23 is represented, likewise in the form of an actual position 54 on the X-axis 47 and an actual position 55 on the Y-axis 48. In FIG. 7, the desired positions 52, 53 are represented by a solid line and the actual positions 54, 55 are represented by broken lines. In reality, different-coloured representations are preferably used in order to identify desired and actual positions. The second step is concluded when the two actual positions 54, 55 correspond to the two desired positions 52, 53 by displacement in a correction direction 56 and 57, respectively.

FIG. 8 shows an alternative screen representation to FIG. 7 for the orientation of the spatial axes of the needle mount 3. In this case, schematic representations 3′ of the needle mount 3 are displayed on the screen 7, together with the corresponding desired and actual positions 52, 53, 54, 55 for X- and Y-axes 47, 48. Experiments have shown that the orientation time required can be reduced again with such a representation.

Overall, a time duration of less than 20 seconds is required for the orientation of the needle mount 3. It is not necessary to mark the entry point 44 on the skin of the patient 5.

In a second exemplary embodiment, a calibration of the needle 6 is required for determining the exact needle length. For this purpose, the position of the needle 6 must be defined with regard to the needle mount 3, on the one hand, and with regard to the reference frame 2, on the other hand. This ensures that needles from different manufacturers can be used.

For this purpose, the user holds the proximal needle end, that is to say the needle tip 58, firstly onto a calibration point 59 on the reference frame 2, wherein the 3D coordinates of said calibration point 59 are made known to the navigation module 36 of the positioning system 100 beforehand or have already been stored in the positioning system 100. A notch or a CT marker 14, the position of which is known to the positioning system 100, advantageously serves as the calibration point 59.

Furthermore, a second DRF (needle DRF) is assigned to the needle 6 itself and calibrated in such a way that the starting point of the needle DRF is situated at the distal needle end 25. For determining the position of the distal needle end 25, optical markers 15 arranged there are used, namely preferably the optical markers 15 connected to the bearing element 24 on the needle mount 3. The needle length is then defined as the length of the vector between the calibration point 59 on the reference frame 2 and the starting point of the needle DRF.

On the basis of the thus known position of the needle 6 in the patient coordinate system 201 and in the coordinate system 301 of the optical tracking system 300, the two coordinate systems can be adjusted by means of the registration module 31 of the positioning system 100.

The actual orientation of the needle mount 3 is effected in two partial steps, as described above. During the needle advance, the needle DRF then moves concomitantly with the needle 6. The exact position of the needle 6, in particular the exact position of the needle tip 58, is determined by the navigation module 36 and is visible in the patient data record on the screen 7. Consequently, a virtual real-time control of the current needle position on the screen 7 is possible.

In order to obtain a control with regard to the actual position of the needle 6 in the body of the patient 5, a CT control scan can be carried out. In this case, the positioning system 100 uses information about the position of the needle 6 within the CT coordinates for proposing to the user a comparatively small region for a CT control scan in the longitudinal direction. The proposed region is advantageously the region around the needle tip 58, since the remaining part of the access path is usually less interesting in this situation. Preferably, corresponding control data are automatically transmitted from the positioning system 100 to the CT scanner 200. Large-area control scans such as are necessary in the solutions known from the prior art primarily in the case of obliquely running intervention trajectories, and which would be associated with a high radiation burden, can be obviated as a result.

If the control scan reveals that a correction of the needle position is necessary, for example because the patient 5 has moved in the meantime, then the new CT data can be used for the further course of the intervention.

In this case, the orientation of the needle 5 and/or the needle advance can be effected automatically, for example with the aid of an orientation and advance device (not represented) that is designed for this purpose and connected to the navigation software for exchanging corresponding data, or else manually by the user. The orientation and advance device is advantageously a robot-based system. The orientation and advance device comprises, for example, a robot module having six degrees of freedom for the orientation of the needle mount and an advance module having servomotors for the needle advance.

During the needle advance, CT control scans can be carried out and the corresponding new CT images can be loaded into the positioning system 100 via the input module 31 in order to check the actual position of the needle 6 and in particular of the needle tip 58. The further needle advance can then be monitored either on the basis of the CT images previously used or else on the basis of the new CT images of the CT control scan.

In addition, during the intervention, screen shots containing information about the last needle position are generated by a further functional module of the navigation software for documentation purposes. Said screen shots are converted into DICOM images by a further functional module of the navigation software and sent to a local image archive, preferably PACS (Picture Archiving & Communication System). Since the PACS is responsible for the archiving and management of the image data, after the intervention all the images and patient data are erased by the positioning system 100.

In a further exemplary embodiment, the positioning system 100 has a calibration member (not illustrated). The latter serves for checking the geometry of the needle mount 3. In particular, the calibration member serves for checking the relative position of rotation point 22 and needle axis 23 with respect to one another. For this purpose, the calibration member itself is exactly measured and the geometry of the calibration member is known to the positioning system 100. Furthermore, optical markers 15 are likewise provided on the calibration member. The calibration member can be provided as an external calibration member. Preferably, however, the calibration member is integrated into the reference frame 2, such that the user can carry out a check of the geometry of the needle mount 3 prior to each application in a simple manner. In this case, the needle mount 3 is brought to a spatial reference with respect to the calibration member in a defined manner. A plug element is preferably provided on the needle mount 3, which plug element can be plugged into a correspondingly provided receptacle opening in the calibration member in a defined manner. The calibration member is preferably integrated into the reference frame 2.

The use of a separate calibration member is not necessary if the reference frame 2 itself is used as a calibration member. Since both the dimensions and the spatial position of the reference frame 2 are known, the reference frame 2 can serve as a calibration member in a simple manner if it has for example a suitable plug element, for example a peg or pin. The spatial arrangement of the plug element is known. The needle mount 3 is then plugged onto the plug element on the reference frame 2. Deviations can be ascertained by means of a comparison of the desired and actual positions of the needle mount 3.

If the positioning system 100 ascertains a deviation in the geometry of the needle mount 3, then the deviation from the desired geometry is calculated by the positioning system 100 and a corresponding correction is effected in the planning of the trajectory 45 or navigation of the needle 6 during the intervention on the basis of a correction matrix determined. In other words, the needle mount 3 is then always used together with the correction matrix. If the deviations exceed a maximum limit value, for example because the needle mount 3 previously fell to the floor, a corresponding message is output to the user or the planned application is terminated by the positioning system 100.

In a further exemplary embodiment, likewise not illustrated, additional optical markers are fitted to the patient in a manner known per se. Said additional optical markers are likewise detected by the optical tracking system 300. For the evaluation of these data, a patient module (not represented) is provided in the computation module 32 of the navigation software, said patient module being designed to recognize changes to the patient 5, in particular movements of the patient 5, with the aid of said data. With the additional optical markers, three essential items of information can be detected, namely whether the patient 5 has moved, how the patient 5 has moved and what position the patient 5 is currently in. These data are preferably used for an automatic real-time correction of the patient data by the positioning system 100. Thus, by way of example, the respiration curve of the patient 5 can be taken into account in the display of the patient data record on the screen 7. Furthermore, these data can also be used in a fully automatic intervention without manual navigation.

The accuracy with which a navigation can take place was determined on the basis of investigations. In this case, trajectories having lengths of 120 mm and 180 mm were planned with the aid of the positioning system 100 according to the invention. A standard biopsy needle (18G) was used. The positioning system 100 calculated the vector v between the current position—determined by the positioning system—of the virtual needle tip, on the one hand, and the planned target point, on the other hand. Furthermore, the positioning system 100 calculated the perpendicular | of the lengthened virtual needle axis to the planned target point. The length e=|v| and the perpendicular k=|l| were used for identifying the error of the incorrect setting. Said error comprises construction errors of needle mount 3 and reference frame 2 as well as errors in the image-to-patient registration and errors caused by the optical tracking system 300.

Table 1 indicates the mean values of the errors with standard deviations, wherein 105 measurements were carried out in each case. The accuracy was therefore distinctly better than 1 mm.

TABLE 1

| Path length [mm] | RMS (e) [mm] | RMS (k) [mm] |
|---|---|---|
| 120 | 0.635 ± 0.228 | 0.481 ± 0.221 |
| 180 | 0.604 ± 0.217 | 0.489 ± 0.204 |

In a further exemplary embodiment, an electromagnetic tracking system (not represented) is used instead of the optical tracking system 300. In this case, marking elements in the form of coils 64 replace the optical markers 15. Said coils 64 are in turn arranged in such a geometry with respect to one another in or on the reference frame 2′, in or on the needle mount 3 and in an instrument, for example the needle 6, that unambiguous determination of the position of these devices is possible if in each case at least one or two coils 64 are detected by the electromagnetic tracking system. In this case, a total of five degrees of freedom result when one coil 64 is used, and six degrees of freedom result when two coils 64 are used. The coils 64 are preferably arranged in such a way that the coil longitudinal axes in each case lie at a right angle to one another. The coils 64 are tracked by means of a field generator that generates an electromagnetic field in the region of the reference frame 2′.

If coils 64 are provided directly in the needle 6, the use of a needle mount can be dispensed with. Orientation and needle advance are then preferably effected by “freehand navigation” by the user. However, the navigation can also be effected in “guided” fashion, for example with the aid of a robot system or a hydraulic arm or the like. It goes without saying that it is also possible to use coils 64 as second marking elements in the needle mount 3.

FIG. 9 represents a further exemplary embodiment of a reference frame 2′ made of plastic, such as can also be used for the positioning system 100 with an electromagnetic tracking system. The reference frame 2′ essentially comprises two bearing arms 61 running parallel to one another, and a shorter central web 62. In this case, bearing arms 61 and central web 62 are embodied in bar-type fashion. The central web 62 is connected to the bearing arms 61 via two planar supporting elements 63, which hold the central web 62 at such a height above the bearing arms 61 that in the free space formed thereby, there is space for a patient 5 completely or at least partially when the reference frame 2 bears with its bearing arms 61 on the CT table 11 of the CT scanner 200. As an alternative to bearing on the CT table 11, the reference frame 2′ can also be positioned above the patient without there being any contact with the CT table 11. Preferably, the reference frame 2′ is then laterally fixed to the CT table 11 with a mounting arm 17 or some other movable holding device.

In a further exemplary embodiment (not represented), the reference frame 2′ is placed onto the patient 5 and optionally fixed to the patient 5 under slight pressure. In this case, the reference frame 2′ simultaneously serves as patient fixing means.

The CT markers 14 are situated within the two bearing arms 61. The coils 64 serving as electromagnetic marking elements are arranged in the supporting elements 63.

One exemplary marker in each case is depicted by broken lines. The optical markers 15 are fitted here on both sides on the central web 63. Two notches in the bearing arms 61 serve as calibration points 59.

The use of an electromagnetic tracking system is particularly advantageous since the coils 64 are comparatively small and can be accommodated without any problems even within the devices (reference frame 2 and needle mount 3), where they are undisturbed relative to all ambient influences. It is furthermore particularly advantageous that such a coil 64 can also be integrated in the needle 6, in particular into the needle tip 58. It thus becomes possible in a simple manner to determine the position of the needle tip 58 in the electromagnetic field with the aid of the positioning system 100 and to display it in real time in the patient data record on the screen 7. During the intervention it can thus be reliably established when the needle tip 58 bends or other changes to the needle tip 58 occur. This is advantageous particularly in the case of very long needles 6, which already have a certain instability on account of their construction.

If the position of the needle tip 58 in the body of the patient 5 can be tracked exactly, a precise advance of the needle 6 on a non-rectilinear trajectory is also possible. This is advantageous particularly when a target point 39 can be accessed only via a non-rectilinear access path, for example when the needle 6 has to be navigated around a bone tissue.

In order to achieve navigation optimized further, it is possible, of course, to combine all the above-described systems and system components with one another in different ways. By way of example, optical markers 15 and electromagnetic markers 64 can be used simultaneously as second marking elements. In a further exemplary embodiment (not represented), by way of example, optical markers 15 are used for identifying the reference frame 2 and the needle mount 3, while electromagnetic markers 64 are used for identifying the needle 6 and thus in particular for tracking the needle tip 58 within the patient's body.

### LIST OF REFERENCE SYMBOLS

1 (Free)
2 Reference frame
3 Needle mount
4 Data processing unit
5 Patient
6 Needle
7 Screen
8 Carrier rack
9 Patient fixing system
10 Personal computer
11 CT table
12 Longitudinal strut
13 Longitudinal direction
14 CT markers
15 Optical markers
16 Receiving and holding device
17 Mounting arm
18 Fixing flange
19 Rotary joint
20 (Free)
21 Bearing rail
22 Rotation point
23 Needle axis
24 Bearing element
25 Distal needle end
26 Serial interface
27 DICOM
28 TCP/IP interface
29 (Free)
30 (Free)
31 Input/output module
32 Computation module
33 Display module
34 Registration module
34a Marker recognition module
34b Adjustment module
35 Planning module
36 Navigation module
37 Target region
38 First tissue type
39 Target point
40 Control unit
41 First entry point
42 First trajectory
43 Second tissue type
44 Second entry point
45 Second trajectory
46 Checkpoint
47 X-axis
48 Y-axis
49 First circle
50 (Free)
51 Second circle
52 Desired position X-axis
53 Desired position Y-axis
54 Actual position X-axis
55 Actual position Y-axis
56 Correction direction X-axis
57 Correction direction Y-axis
58 Needle tip
59 Calibration point
60 (Free)
61 Bearing arm
62 Central web
63 Supporting element
64 Coil
100 Positioning system
200 CT scanner
201 First coordinate system
300 Tracking system
301 Second coordinate system

1-19. (canceled)

20. A positioning system for percutaneous interventions, comprising:
a reference frame being arranged in a defined position relative to a patient, said reference frame being configured to determine the position thereof in a first reference system and in a second reference system,
an instrument mount for receiving a medical instrument, at least one of said instrument mount and said medical instrument being positioned in said second reference system, and
a data processing unit having
a) an input module for receiving a patient data record provided by an imaging system and for receiving an apparatus data record provided by a tracking system,
wherein the patient data record contains patient data, including image data, in said first reference system and data regarding the position of said reference frame in said first reference system,
and wherein the apparatus data record contains data regarding the position of at least one of said reference frame and said medical instrument in said second reference system and data regarding the position of at least one of said instrument mount and said medical instrument in said second reference system,
b) a registration module for performing an automatic image-to-patient registration using the data contained in the patient data record and in the apparatus data record, and
c) a planning module for planning a trajectory from an entry point on the patient to a target point in the patient.

21. The positioning system according to claim 20, wherein said data processing unit includes
a navigation module to visualize at least one of said instrument mount and said medical instrument in the patient data record before and/or during intervention.

22. The positioning system according to claim 20, wherein said reference frame includes a plurality of first marking elements to determine its position in said first reference system and a plurality of second marking elements to determine its position in said second reference system.

23. The positioning system according to claim 20, wherein said instrument mount includes a plurality of second marking elements to determine its position in said second reference system.

24. The positioning system according to claim 20, wherein said medical instrument includes a plurality of second marking elements to determine its position in said second reference system.

25. The positioning system according to claim 22, wherein said first marking elements determine a position in said first reference system with the aid of said imaging system.

26. The positioning system according to claim 22, wherein said second marking elements determine a position in said second reference system with the aid of at least one of an optical and electromagnetic tracking system.

27. The positioning system according to claim 22, wherein said registration module performs at least one of the following: identifies the position of said first marking elements in said first reference system, and identifies the position of said second marking elements in said second reference system and adjusts said first reference system with said second reference system based on the positions of said first and second marking elements.

28. The positioning system according to claim 20, wherein said imaging system is one of a computer tomograph and a C-arc system.

29. The positioning system according to claim 20, wherein said tracking system is at least one of an optical and electromagnetic tracking system.

30. The positioning system according to claim 20 is part of a patient fixing system to fix the patient, said system operates on a vacuum principle.

31. The positioning system according to claim 20, wherein said reference frame is arranged in a defined position relative to the patient, said reference frame having a plurality of first marking elements to determine its position in said first reference system and a plurality of second marking elements to determine its position in said second reference system, said first marking elements determine the position with the aid of an imaging system, and said second marking elements determine the position with the aid of at least one of an optical and electromagnetic system.

32. The positioning system according to claim 31, wherein said reference frame can be fitted with respect to the patient.

33. The positioning system according to claim 31, wherein said reference frame is an integrated calibrating member.

34. The positioning system according to claim 20, wherein said instrument mount has a receiving device to secure said medical instrument, and comprises a plurality of second marking elements to determine its position in said second reference system, and said second marking elements determine its position with the aid of at least one of an optical and electromagnetic tracking system.

35. The positioning system to claim 34, wherein said instrument mounts comprises rotary joints to orient a rotation point of said instrument mount and an axis of said medical instrument.

36. The positioning system according to claim 20, wherein said medical instrument has a plurality of second marking elements to determine its position in said second reference system, said second marking elements determine its position with the aid of at least one of an optical and electromagnetic tracking system.

37. The positioning system according to claim 20, wherein said data processing unit is a computer which generates computer instructions,
the computer program instructions receive the patient data record and apparatus data record,
the computer program instructions carry out an automatic image-to-patient registration with the aid of the data contained in the patient data record and in the apparatus data record, and
the computer program instructions plan a trajectory from an entry point on the patient to a target point in the patient.

38. The positioning system according to claim 37, wherein
the computer program instructions visualize at least one of said instrument mount and said medical instrument in the patient data record at least one of before and during the intervention.
